(19) United States
(12) Patent Application Publication
(10) Pub. No.: US 2009/0105641 A1
(43) Pub. Date: Apr. 23, 2009

(54) BALLOON DILATATION CATHETER

(76) Inventor: Thomas Nissl, Garstedt (DE)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/996,506

(22) PCT Filed: Jul. 24, 2006
(86) PCT No.: PCT/EP2006/007266

§ 371 Date: Jul. 16, 2008

(30) Foreign Application Priority Data
Jul. 23, 2005 (DE) 10 2005 034 529.8

(51) Int. Cl. A61M 29/02

(52) U.S. Cl. 604/97.02; 604/101.02; 604/96.01; 604/103.09

(57) ABSTRACT

Embodiments of the present invention relate to a balloon dilatation catheter, in particular for percutaneous transluminal coronary angioplasty (PTCA), provided with a guide wire, a supply tube extending parallel to the guide wire and serving for inflating a dilatation balloon, as well as the dilatation balloon arranged in the distal area of the supply tube, wherein the dilatation balloon has a tubular configuration with a wall structure comprising a plurality of tube segments arranged adjacent to each other of which at least one is connected with the supply tube.

The invention relates to a balloon dilatation catheter, in particular for percutaneous transluminal coronary angioplasty (PTCA), provided with a guide wire, a supply tube extending parallelly to the guide wire and serving for inflating a dilatation balloon, as well as the dilatation balloon arranged in the distal area of the supply tube.

The inventive balloon dilatation catheter is primarily meant for the percutaneous transluminal coronary angioplasty (PCTA) but may also be employed for the widening of other, especially peripheral blood vessels. Moreover, the catheter can also be used in other body lumina.

Balloon dilatation catheters are frequently employed for the treatment of strictures, stenoses or constrictions in other parts of the human body. Balloon catheters of this type are for example intended for percutaneous transluminal angloplasty (PTA) and especially in the coronary area (PTCA) where they serve to eliminate vascoconstrictions. For this purpose, a dilatation balloon is transferred through an intravascularly extending catheter to the constricted location where said balloon is inflated hydraulically via a supply tube to expand the constriction and, during the dilatation process, compress any existing deposits such that the lumen of the vessel remains expanded at least to a great extent after the balloon has been removed from the vessel, although it is understood that it is normally impossible as a rule to completely restore the originally existing vessel lumen.

Balloon techniques applied for the dilatation of a vessel are in competition to intraluminal stents insofar as the stents also serve vessel dilatation purposes and usually remain at the implantation site where they have a permanent expansion effect. The fact that dilatation often lacks permanence is viewed as a significant disadvantage in the treatment of stenoses with balloon catheters.

The reason for the lack of dilatation permanence when using a dilatation balloon is associated with the tendency of body vessels to re-contract when the pressure exerted by the balloon is eliminated. This is primarily due to the short time span during which a dilatation balloon is allowed to take effect. Since dilatation balloons of customary design result in a more or less complete vessel obliteration such a permanency improvement, i.e. a prolonged residence time, cannot be achieved as a rule. This is one of the reasons why attending physicians often prefer the use of stents to balloon dilatation methods.

On the other hand, stents remain in the vessel as a foreign object after treatment has been completed whereas dilatation balloons are removed.

Trials were conducted with catheters that had a greater lumen and thus allowed blood flow to a certain extent; however, the blood passage attainable with these catheters was greatly restricted as a rule. Catheters with blood pumps have also been developed but proved to be of sophisticated design and expensive, with the blood volume that could be transferred still remaining low and, additionally, with a risk of blood impairment existing.

It is thus the objective of the invention to provide a balloon system for vessel dilatation purposes that enables both the widening of a vessel wall and, at the same time, maintaining an adequate flow of blood through the expanded location and in this way achieve a longer system residence time. The same applies to the use of the system in other body lumina.

This objective is achieved by providing a balloon dilatation catheter of the kind first mentioned above wherein the dilatation balloon has a tubular configuration with a wall structure comprising a plurality of tube segments arranged adjacent to each other of which at least one is connected with the supply tube.

The inventive balloon dilatation catheter differs from those of customary type in that a special design of the balloon portion has been provided. Otherwise, the catheter according to the invention has been designed in a manner known per se.

The tubular design of the dilatation balloon is brought about due to the fact that the wall structure is formed by a plurality of tube segments arranged adjacent to each other. The pressure medium is led to these tube segments via the supply tube, and it will suffice to provide and make up a single connection with the supply tube in case the tube segments are interconnected. As an alternative, all tube segments may have a connection to the supply tube and may thus be filled with the pressure medium individually. Combinations of the two variants are also possible.

The tube segments of the balloon wall structure are essentially of parallel arrangement and form an inner surface defining the remaining duct or central passage as well as an outer surface pressing against the vessel wall and thus expanding it after the pressure medium has been admitted/injected.

At the contact lines the tube segments connect to each other to enable the outer surface of the balloon to have an as homogeneous as possible form and for this purpose they are stuck together or welded at said contact lines. This connection method ensures that the balloon is uniformly dilatated when pressure is applied.

As per a variant of the dilatation balloon used as proposed in accordance with the invention the tube segments of the balloon are designed to be of circular shape. They are of parallel arrangement and essentially project perpendicularly from the central supply tube. This embodiment in particular provides for each individual tube segment to be connected to the supply tube and have a circular configuration starting out from the supply tube and extending back to it. It is not necessary to provide for a double connection to the supply tube; it is, however, thought expedient to attach the respective dead end of each tube segment to the supply tube, in particular by welding.

As per another embodiment the tube segments are designed so as to form windings of a helically wound tube, with said windings also being stuck together or welded at the lines where they are in contact with each other. At the distal end the helically wound tube is closed off and at its proximal end connected to the supply tube. In this case it is sufficient to arrange for a single connection to the supply tube through which the pressure medium can be introduced into the entire tubular balloon.

As provided for by another embodiment the catheter according to the invention may accommodate in its interior space an inner balloon in such a manner that the dilatation balloon of tubular shape embraces this inner balloon. This inner balloon is connected to a separate supply tube and can be guided separately.

This second or inner balloon serves as an expansion aid for the tubular balloon, especially to prevent sagging or buckling that might occur during the dilatation process. For this purpose, expansion of the inner balloon takes place at the same time as that of the tubular balloon, but said balloon may also be expanded beforehand or subsequently in order to correct the configuration of an incompletely unfolded tubular balloon. After the expansion of the tubular balloon has been completed the inner balloon is retracted, for example by means of a separate guide wire or via the supply tube. Retracting the inner balloon clears the interior space of the tubular dilatation balloon and allows the free flow of blood.

The catheter in accordance with the invention is provided with an essentially conventional guide wire extending within the catheter parallelly to the supply tube and inside the tubular dilatation balloon. Preferably, it is secured at the inner wall of the balloon, especially preferred at the side of the balloon inner wall opposite the supply tube.

Balloon and/or guide wire may be provided in a customary manner with markers at the proximal and/or distal ends. It is thought expedient as a rule to arrange the markers on the guide wire such that the balloon can be precisely positioned with the aid of these markers.

Catheter, guide wire, supply tube as well as balloons consist of materials customarily used for the purposes referred to herein which for the tubular balloon and the inner balloon usually are PET, polyamide, polyolefin, polyurethane or polyester, with PET and polyamide being preferred. It is expedient to design the balloon to be more flexible and expansible than the supply tube; however, the stabilization of the supply tube wall structure to withstand pressure forces may also be achieved by providing a catheter highly resistant to pressure.

The inventive balloon dilatation catheter is particularly suited for the percutaneous transluminal coronary angioplasty but may also be employed for the widening of other blood vessels. Furthermore, it may be employed in applications involving urology, bronchial tubes, intestinal diseases, esophagus, bile ducts and trachea. Manipulation, handling and application conditions coincide with those applicable to customary balloon catheters.

The advantage of the inventive balloon dilatation catheter is that it may be left in the vessel for a longer time span without the risk of blocking the vessel function fundamentally. In this manner blood vessels can be dilatated for a longer period ranging from several minutes to several hours which counteracts the tendency of body vessels to contract when the balloon has been removed. In this respect, the tubular balloon employed as proposed by the invention fulfills the function and has the effect of a temporary stent.

If appropriately designed, the tubular balloon of the inventive balloon dilatation catheter may also be used as stent. This necessitates that the balloon dilatation catheter is provided with a one-way valve enabling the tubular balloon to be irreversibly inflated, with said valve being arranged in the portion of the supply tube immediately adjacent to the tubular balloon. As soon as the balloon has reached its targeted size the fluid supply is terminated and the supply tube detached at a point immediately proximal to the one-way valve. This may be achieved, for example, by cutting off using a hot wire sling, by providing a bayonet or screw connection or with the aid of a predetermined breaking point that can be activated.

A tubular balloon isolated in this way may, for example, be used for the dilatation of body lumina that cannot be widened by means of customary stents. The balloon acts as a stent that due to its flexible, soft surface only exerts a moderate pressure on the surrounding tissue. It is therefore suitable for application in areas of benign tumors and vegetations where customary stents are contraindicated.

For the elimination of such a tubular balloon used as stent it will be sufficient to broach or pierce it and remove the pressureless envelope with the help of a retriever.

Accordingly, the invention also relates to a tubular balloon that having been detached from the inventive balloon dilatation catheter in the manner described hereinbefore can be placed in a body lumen where it serves as stent. Such a tubular balloon has all the characteristics described herein in connection with the inventive balloon dilatation catheter. Such a tubular balloon is of course additionally provided with the above described one-way valve and, as the case may be, the attachment element of the cut-off supply tube. Regarding the description of the tubular balloon reference may thus be made to the description of the catheter-bound tubular balloon herein.

The invention is explained in more detail by way of the enclosed figures where

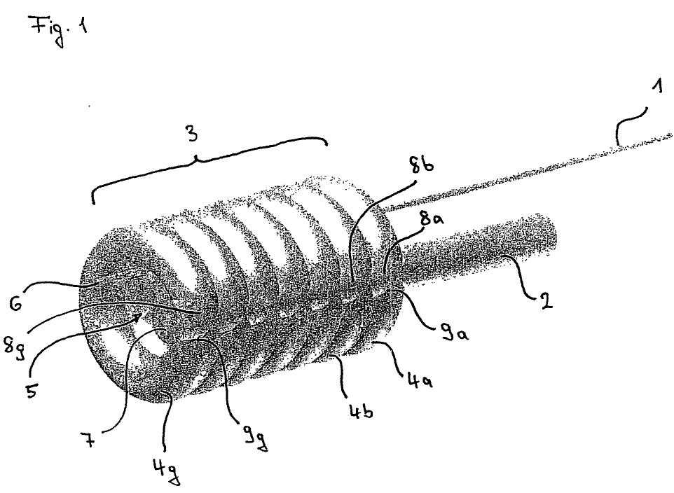

FIG. 1: shows a first embodiment of a tubular balloon in accordance with the invention;

FIG. 2: is a schematic sectional view through a tubular balloon according to FIG. 1 in longitudinal direction;

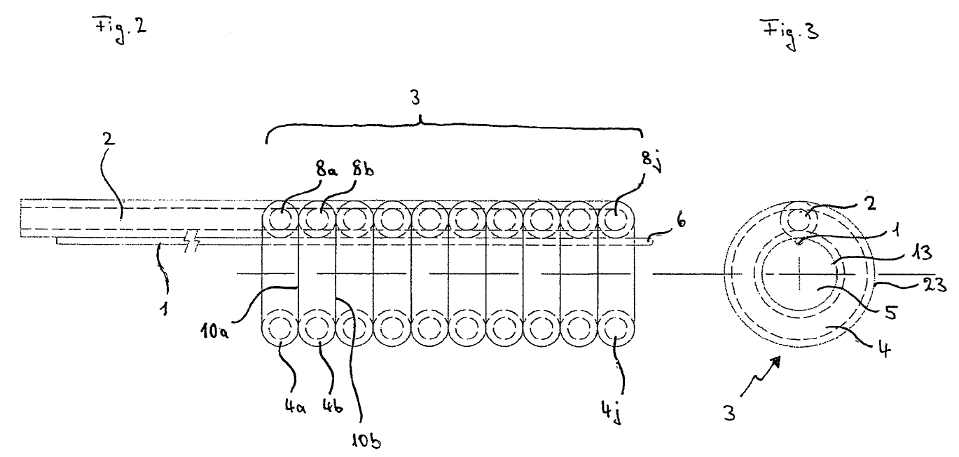

FIG. 3: is another sectional view taken at right angles to the supply tube extension;

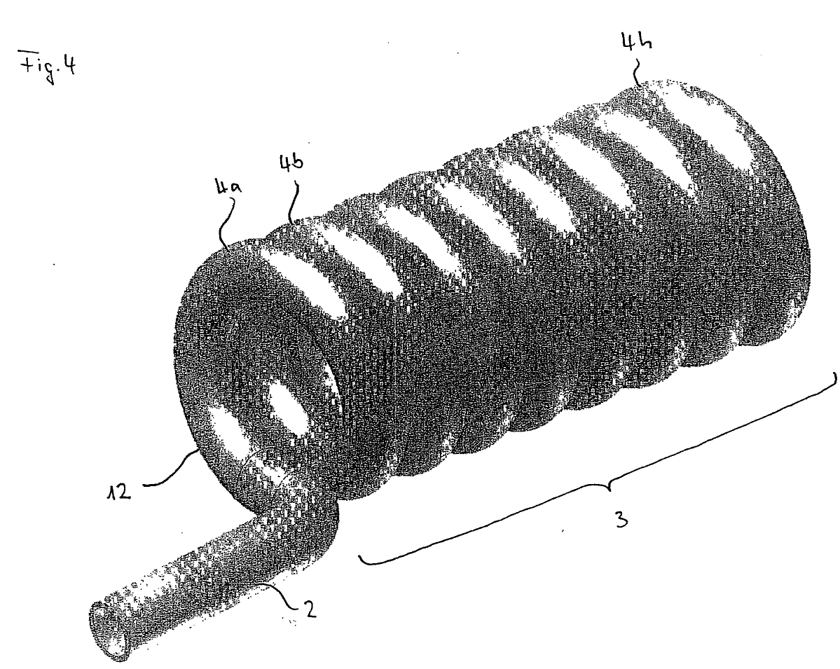

FIG. 4: is a second variant of a tubular balloon in accordance with the invention;

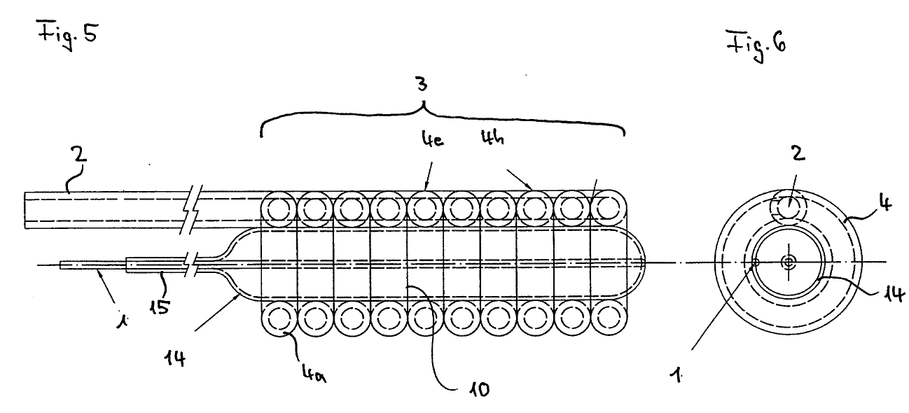

FIG. 5: shows a third variant of a tubular balloon as per the invention provided with an inner balloon;

FIG. 6: is a cross-sectional view of the inventive tubular balloon as per FIG. 5;

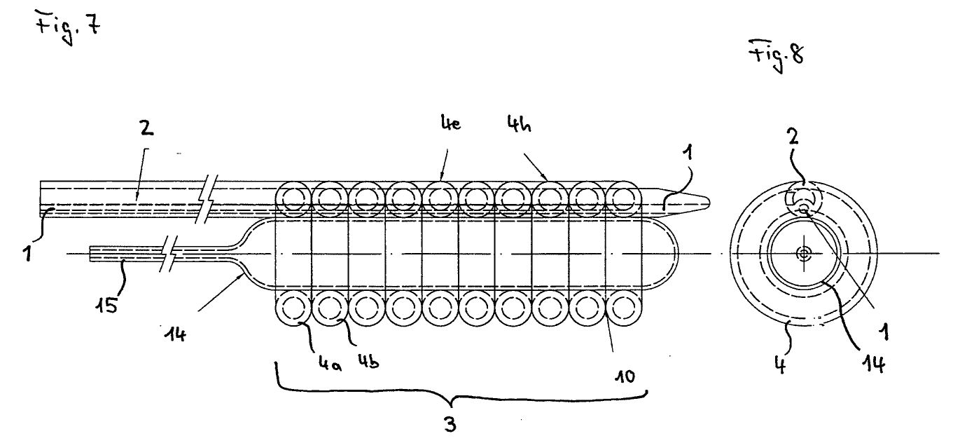

FIG. 7: shows a fourth variant of a tubular balloon as per the invention provided with an inner balloon; and

FIG. 8: shows a cross-sectional view of the balloon as per FIG. 7.

FIG. 1 shows a first embodiment of the invention with the (customary) catheter not being included in the representation.

The illustration depicts guide wire 1 with its distal end 6 as well as the supply tube 2 which serves for the pressurizing of the balloon (3) by means of a hydraulic pump through the catheter not shown here. The supply tube 2 is closed off at its distal end 7.

The dilatation balloon 3 is of tubular design and enables an inner passage or hollow space 5 to be formed which remains cleared, especially when the balloon is inflated, and permits body fluids to pass through. The wall structure of the tubular balloon 3 consists of a plurality of tubular segments 4 (4a, 4b . . . 4g) arranged parallelly to and in contact with each other. Tube segments 4 start out from the supply tube 2 and are attached to said tube via connections 8 (8a, 8b . . . 8g) such that they can be pressurized. They extend from the supply tube 2, form a circle and extend back to said supply tube 2; however, at their respective opposite ends they have no connection with supply tube 2 but dead-end there. Nevertheless, these ends 9 (9a, 9b . . . 9g) are permanently attached to the supply tube 2 with the aid of bonding or welding methods. Alternatively, ends 9 may also be connected with the supply tube 2.

The individual tube segments 4 also connect to each other at the contact lines, i.e. they are bonded or welded together. This means at its contact line tube segment 4a is welded to tube segment 4b, tube segment 4b at its contact line to tube segment 4c etc. The tube segments form a closed wall structure and are pressurized centrally via the supply tube, in each case through connection points 8a, 8b, . . . 8g.

The tubular balloon illustrated in FIG. 1 may, for example, be manufactured in that the respective tubular segments 4 are welded or connected to the supply tube 2 parallel to each other after the supply tube 2 has been provided with apertures at the relevant locations. The supply tube 2 is closed off at its distal end. Following this, the dead ends 9 of the tube segments 4 are also attached to the supply tube 2 by bonding or welding.

The balloon may be advantageously manufactured in that two balloon tubes are laid out in duplicate and welded together at the proximal and distal end. At the proximal end the feed element, the supply tube 2, through which pressurizing is effected is welded in. Over the length of the balloon radial welds are made at defined intervals so that individual tube segments are formed. In continuation of the welded-in supply tube 2 a “flooding duct” (inflation lumen) is left open, i.e. no welds are arranged in this area.

Due to the fact that the basic tube is everted or folded back on itself for manufacturing purposes one welding seam, preferably the distal weld, can be saved.

The wall thickness of the tubular balloon is determined by the spacing of the radial welds. This determines the diameter of the individual, radially extending tubes which in general ranges between 0.5 and 5 mm as needed for the respective application.

FIG. 2 is a schematic sectional view in longitudinal direction through a tubular balloon as shown in FIG. 1. The supply tube 2, the wall thickness of which is shown in broken lines, extends in parallel to the guide wire 1.

From the supply tube 2 at locations eight tube segments 4 branch off said tube in its distal area which corresponds to the length of the tubular balloon 3, with said tube segments being pressurized via the supply tube 2. The tube segments 4 are welded to each other at contact lines 10. This means that the tube segments 4a and 4b are welded together at contact line 10a, the tube segments 4b and 4c at contact line 10b.

Guide wire 1 extends through the inner space of the tubular balloon 3 and with its distal end 6 projects beyond the tubular balloon 3. Expediently, it is secured to the inside of the tubular balloon 3. In the area of its distal end 6 and immediately proximal to balloon 3 the guide wire may be provided with radiopaque markers. Alternatively or additionally, markers may be arranged on the tubular balloon at the proximal and distal ends.

FIG. 3 is a sectional view taken at right angles to the extension of the supply tube of the embodiment shown in FIGS. 1/2. The sectional drawing shows the tubular balloon 3 with its inner wall structure 13 and its outer wall structure 23 as well as the inner free space, shown delineated by broken lines, which is to be filled with the pressure medium. The pressure medium is fed in through the supply tube 2 leading to the pump. Guide wire 1 extends along the inner wall 13 to which it adjoins and is attached.

FIG. 4 shows a second variant of the tubular balloon to be used in accordance with the invention, with said balloon comprising the individual tube segments 4a, 4b, . . . 4h and part and in the form of a helically wound tube 12. The individual windings 4a, 4b, . . . 4h are welded together at the contact locations in the same manner as provided for by the other embodiment. At its proximal end tube 12 of the tubular balloon 3 directly merges into the supply tube 2, and said supply tube 2 may consist of the same material or of a material that has other expansion characteristics.

FIG. 5 shows a third variant of a tubular balloon 3 to be used in accordance with the invention, said balloon accommodating in its interior (hollow space) an inner balloon 14. As illustrated above, the tubular balloon 3 consists of individual tube segments 4a . . . 4e, . . . 4h that are welded together at their relevant contact lines 10. Tubular balloon 3 is connected to a supply tube 2 of a design as described hereinbefore. The inner balloon 14 has a separate supply tube 15 which serves for pressurizing purposes. The guide wire 1 extends along the inner wall of tubular balloon 3 and over the outer wall of the inner balloon 14, refer to FIG. 6.

FIG. 6 is a cross-sectional representation of the embodiment shown in FIG. 5. The tube segment 4 embraces the inner balloon 14 concentrically and has direct contact with the guide wire 1 extending along the inner wall structure of tubular segments 4. Tube segments 4 are in connection with the supply tube 2.

In FIG. 7 a fourth embodiment of the invention is illustrated and shows tubular balloon 3 which is expanded via the supply tube 2, with guide wire 1 extending through the interior of said balloon. The individual tube segments 4a, 4b, . . . 4e, . . . 4h are welded to each other at contact locations 10. Inside the tube segments 4 an inner balloon 14 is arranged and provided with its own supply tube 15, When inflated, said inner balloon 14 causes the tubular balloon 3, i.e. the segments 4 of tubular balloon 3, to expand and in this way assists their inflation process.

FIG. 8 is a cross-sectional representation of the embodiment illustrated in FIG. 7 showing tube segment 4, inner balloon 14 and supply tube 2. The guide wire 1 extends through a separate compartment of the supply tube 2 immediately adjacent to the inner space of tube segments 4, with said inner space being filled with inner balloon 14.

The variants illustrated in FIG. 5 to 8 show the inventive dilatation catheter with inner balloon 14 in place. It is understood that this inner balloon 14 only serves the purpose of providing assistance with a view to bringing about the full expansion of the tubular balloon 13. After tubular balloon 3 has been fully expanded the inner balloon is de-pressurized and can be retracted and removed from the inner space of tubular balloon 3 thus clearing said inner space and allowing blood to pass through.

That which is claimed:

1-18. (canceled)

19. A balloon dilatation catheter comprising:
a dilatation balloon having a tubular configuration with a wall structure comprising a plurality of tube segments arranged adjacent to each other; and
a supply tube configured to inflate the dilatation balloon, wherein the dilatation balloon is arranged in a distal area of the supply tube, and wherein at least one of the plurality of tube segments is coupled to the supply tube.

20. The catheter according to claim 19, wherein adjacent tube segments are connected to each other along respective contact lines.

21. The catheter according to claim 20, wherein adjacent tube segments are welded to each other along respective contact lines.

22. The catheter according to claim 19, wherein each of the plurality of tube segments is circular in shape and has opposed ends.

23. The catheter according to claim 22, wherein at least one of the opposed ends of each of the plurality of tube segments is coupled to the supply tube.

24. The catheter according to claim 19, wherein each of the plurality of tube segments is connected to the supply tube via a respective connection.

25. The catheter according to claim 19, wherein the plurality of tube segments comprise a plurality of windings of a helically wound tube.

26. The catheter according to claim 25, wherein the helically wound tube is connected to the supply tube via a single connection.

27. The catheter according to claim 19, further comprising an inner balloon disposed within the dilatation balloon and connected to a respective supply tube configured to inflate the inner balloon.

28. The catheter according to claim 27, wherein the inner balloon comprises a guide wire.

29. The catheter according to claim 19, further comprising a guide wire extending within the dilatation balloon and parallel to the supply tube.

30. The catheter according to claim 29, wherein the wall structure of the dilatation balloon comprises an inner wall structure and an outer wall structure, and wherein the guide wire is attached to the inner wall structure.

31. The catheter according to claim 30, wherein the supply tube is located radially opposite the guide wire.

32. The catheter according to claim 29, wherein the guide wire and/or dilatation balloon each comprises markers arranged at respective proximal and/or distal ends.

33. The catheter according to claim 19, wherein the dilatation balloon comprises a polyethylene terephthalate, polyamide, or polyolefin material.

34. The catheter according to claim 19, wherein the dilation balloon and supply tube comprise respective materials having different expansion characteristics.

35. The catheter according to claim 19, wherein the supply tube and dilatation balloon comprise pressure resistant material.

36. The catheter according to claim 19, wherein the supply tube comprises a one-way valve arranged proximal to the dilatation balloon and configured to prevent deflation of the dilatation balloon.

37. A method for the dilating a vessel comprising:
delivering a dilatation balloon and a supply tube coupled to the dilatation balloon within the vessel, the dilatation balloon having a tubular configuration with a wall structure comprising a plurality of tube segments arranged adjacent to each other; and
injecting a pressure medium into the supply tube to inflate the dilatation balloon and dilate the vessel.

38. The method according to claim 37, further comprising inserting an inner balloon within the dilatation balloon and injecting a pressure medium into a separate supply tube coupled to the inner balloon to inflate the inner balloon.

39. The method according to claim 38, further comprising retracting the inner balloon and respective supply tube from within the dilatation balloon after inflating the inner balloon.
